(19) United States
(12) Patent Application Publication
(10) Pub. No.: US 2024/0115149 A1
(43) Pub. Date: Apr. 11, 2024

(54) METHOD FOR DETERMINING AN EQUIVALENT SPEED, APPARATUS, DEVICE, AND STORAGE MEDIUM

(71) Applicant: Guangdong COROS Sports Technology Joint Stock Company, Dongguan (CN)

(72) Inventors: Yang YANG, Dongguan (CN); Xin LIU, Dongguan (CN)

(21) Appl. No.: 18/449,919

(22) Filed: Aug. 15, 2023

(30) Foreign Application Priority Data
Sep. 29, 2022 (CN) 202211204035.8

(51) Int. Cl. A61B 5/024; A61B 5/00

(52) U.S. Cl. CPC A61B 5/024; A61B 5/6802; A61B 2560/0252; A61B 2560/0257

(57) ABSTRACT

Provided are a method for determining an equivalent speed, an apparatus, a device, and a storage medium. The method includes the steps described below. The heart rate-speed relationship of a user in a standard environment is acquired. A first heart rate and a first speed of the user are acquired. An equivalent speed of the user in the standard environment is determined according to the first heart rate, the first speed, and the heart rate-speed relationship. The method for determining an equivalent speed disclosed in the present invention converts the current speed of a user into an equivalent speed in a standard environment by using the heart rate-speed relationship in the standard environment.

### CROSS-REFERENCE TO RELATED APPLICATION(S)

This application claims priority to Chinese Patent Application No. 202211204035.8 filed Sep. 29, 2022, the disclosure of which is incorporated herein by reference in its entirety.

### TECHNICAL FIELD

The present invention relates to the field of smart wearable devices and, in particular, to a method for determining an equivalent speed, an apparatus, a device, and a storage medium.

### BACKGROUND

Currently, smart wearable devices are widely used in sports, including trail running and marathon running. For features such as portability, positioning, navigation, and training guidance, smart wearable devices are increasingly used by sports enthusiasts. In sports, particularly running-related sports, speed is an important concept, that is, speed is the mileage per hour of a runner. A runner's endurance level can be accurately reflected by the heart rate-speed relationship, by which the runner is guided in training and competition.

However, when conditions for outdoor sports become harsh, where factors such as air temperature, altitude, slope, and weather are involved, the heart rate-speed relationship in a physical sense acquired by satellite positioning becomes complex and unpredictable. Therefore, the speed in the conventional sense, that is, the speed acquired through satellite positioning, is limited in evaluating or directing the runner to exercise, particularly under the influence of a large number of complex environmental factors.

### SUMMARY

The invention provides a method for determining an equivalent speed, an apparatus, a device, and a storage medium, so as to convert a user's real-time speed into an equivalent speed in a standard environment.

According to an aspect of the present invention, a method for determining an equivalent speed is provided. The method includes the steps described below.

A heart rate-speed relationship of a user in a standard environment is acquired.

A first heart rate and a first speed of the user are acquired.

An equivalent speed of the user in the standard environment is determined according to the first heart rate, the first speed, and the heart rate-speed relationship.

According to another aspect of the present invention, an apparatus for determining an equivalent speed is provided. The apparatus includes at least one processor for performing the following steps of:

A heart rate-speed relationship of a user in a standard environment is acquired.

A first heart rate and a first speed of the user are acquired.

An equivalent speed of the user in the standard environment is determined according to the first heart rate, the first speed, and the heart rate-speed relationship.

According to another aspect of the present invention, an electronic device is provided. The electronic device includes at least one processor and a memory.

The memory is in a communication connection with the at least one processor.

The memory stores a computer program executable by the at least one processor. The computer program is configured to, when executed by the at least one processor, cause the at least one processor to execute the method for determining an equivalent speed described in any embodiment of the present invention.

According to another aspect of the present invention, a non-transitory computer-readable storage medium is provided, which is configured to store computer instructions for implementing the method for determining an equivalent speed described in any embodiment of the present invention when the computer instructions are executed by a processor.

Embodiments of the present invention provide a method for determining an equivalent speed. The method includes the steps described below. First, a heart rate-speed relationship of a user in a standard environment is acquired. Then a first heart rate and a first speed of the user are acquired. Finally, an equivalent speed of the user in the standard environment is determined according to the first heart rate, the first speed, and the heart rate-speed relationship. The method for determining an equivalent speed disclosed in the present invention converts the current speed of a user into an equivalent speed in a standard environment by using the heart rate-speed relationship in the standard environment. In this manner, the speed data conforms to the physiological intensity of the user in the current environment, the delay of changes in the human heart rate is avoided, and the user is assisted more effectively to control the intensity of exercise or change the strategy of a competition in a complex external environment.

It is to be understood that the contents described in this part are not intended to identify key or important features of embodiments of the present invention and are not intended to limit the scope of the present invention. Other features of the present invention are readily understood through the description hereinafter.

### BRIEF DESCRIPTION OF DRAWINGS

To illustrate solutions in embodiments of the present invention more clearly, the accompanying drawings used in description of the embodiments are briefly described below. Apparently, the accompanying drawings described below illustrate part of embodiments of the present invention, and those of ordinary skill in the art may obtain other accompanying drawings based on the accompanying drawings described below on the premise that no creative work is done.

FIG. 1 is a flowchart of a method for determining an equivalent speed according to an embodiment of the present invention.

FIG. 2 is a flowchart of a method for determining an equivalent speed according to an embodiment of the present invention.

FIG. 3 is a diagram illustrating the process of a method for determining an equivalent speed according to an embodiment of the present invention.

FIG. 4 is a diagram illustrating the structure of an electronic device implementing the method for determining an equivalent speed according to an embodiment of the present invention.

### DETAILED DESCRIPTION

The solutions in embodiments of the present invention are described clearly and completely in conjunction with drawings in the embodiments of the present invention from which the solutions are better understood by those skilled in the art. Apparently, the embodiments described below are part, not all, of the embodiments of the present invention. Based on the embodiments described herein, all other embodiments obtained by those skilled in the art on the premise that no creative work is done are within the scope of the present invention.

It is to be noted that terms such as “first” and “second” in the description, claims, and drawings of the present invention are used to distinguish between similar objects and are not necessarily used to describe a particular order or sequence. It should be understood that the data used in this manner are interchangeable where appropriate so that the embodiments of the present invention described herein may also be implemented in a sequence not illustrated or described herein. In addition, terms “comprising”, “including”, and any other variations thereof are intended to encompass a non-exclusive inclusion. For example, a process, method, system, product, or device that includes a series of steps or units not only includes the expressly listed steps or units, but may also include other steps or units that are not expressly listed or are inherent to such a process, method, product, or device.

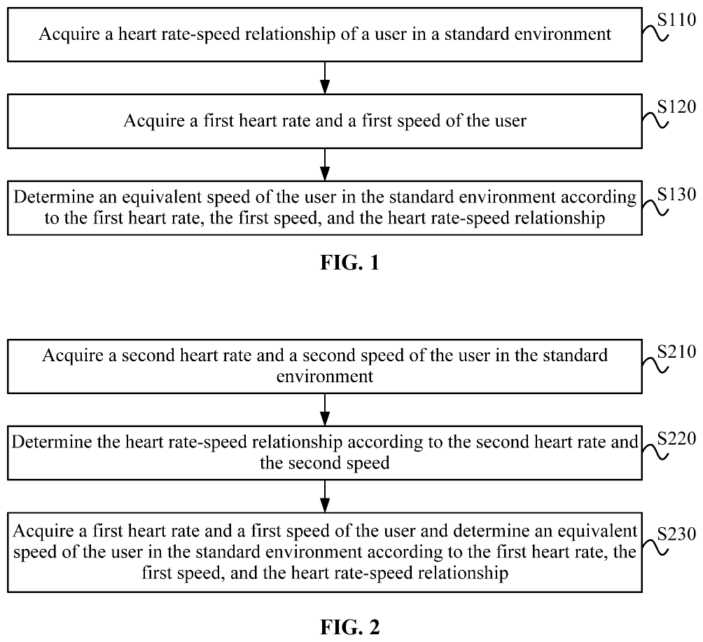

FIG. 1 is a flowchart of a method for determining an equivalent speed according to an embodiment of the present invention. This embodiment is applied to convert a user's real-time speed into an equivalent speed in a standard environment. The method may be executed by an apparatus for determining an equivalent speed. The apparatus may be implemented in the form of software and/or hardware. The apparatus may be configured in an electronic device. As shown in FIG. 1, the method includes the steps described below.

S110: A heart rate-speed relationship of a user in a standard environment is acquired.

The user denotes an object of heart rate and speed monitoring using the method for determining an equivalent speed. The standard environment may denote a stable conventional environment. The heart rate denotes the number of heart beats per minute. The speed denotes the current exercise speed of the user. The heart rate-speed relationship may denote a corresponding relationship between the user's heart rate and speed.

For example, the standard environment may be set as temperature (T (° C.)), slope (S), and altitude (H (m)) that satisfy the following conditions: 10° C.≤T≤28° C., −3%<S<3%, and H<1200 m.

In this embodiment, when the user is in different environments, the heart rate-speed relationship may be different. The heart rate-speed relationship in the standard environment may be acquired by the following method: When the user is in the standard environment, the heart rate data and the speed data of the user are continuously acquired and then fitted to acquire the heart rate-speed relationship of the user in the standard environment. It is also possible to preset the heart rate-speed relationship in the standard environment in the system that is in line with most users' conditions.

Optionally, the heart rate-speed relationship is preset by a system.

Specifically, data may be collected in the early stage to acquire the heart rate data and speed data of a large number of users in the standard environment. The heart rate-speed relationship in the standard environment suitable for most users can be determined by means of function fitting and preset in the system.

S120: A first heart rate and a first speed of the user are acquired.

The first heart rate and the first speed denote the heart rate data and speed data acquired in real time for the current user.

In this embodiment, the first heart rate and the first speed of the user may be acquired separately by a corresponding sensor. Preferably, the sensor may be disposed on a wearable device worn by the user and continuously acquires real-time data at a set working frequency.

S130: An equivalent speed of the user in the standard environment is determined according to the first heart rate, the first speed, and the heart rate-speed relationship.

In this embodiment, the heart rate-speed relationship denotes the heart rate-speed relationship when the user is in the standard environment. However, the environment where the user is located may not be the standard environment when the first heart rate and the first speed are acquired. For example, in the case where the user runs on an uphill road, when the user reaches the same heart rate value, compared with a case where the user runs on flat ground, the speed of running on the uphill road is generally lower than that of running on the flat ground. Therefore, if the user is in a non-standard environment, the corresponding speed data may not have guiding significance. The equivalent speed denotes the speed value converted into the standard environment from the acquired user's current speed data.

Optionally, to obtain accurate speed data, the first speed may be substituted into the heart rate-speed relationship to obtain the equivalent heart rate in the standard environment. Then, the ratio of the first heart rate and the equivalent heart rate is used as an equivalent coefficient between the user's current environment and the standard environment. The product of the first speed and the equivalent coefficient is determined as the equivalent speed of the user in the standard environment.

For example, the heart rate-speed relationship is that HR=k×speed+b. HR denotes the heart rate (bmp). Speed denotes the speed (m/s). k and b are fitting coefficients. Assuming that the first heart rate and the first speed are HR₁ and speed₁ respectively, speed₁ is substituted into the heart rate-speed relationship, and the equivalent heart rate HR₁′ can be obtained. Then the ratio of the first heart rate to the equivalent heart rate is that

$${E}_{1} = \frac{{HR}_{1}}{{HR}_{1}^{′}} ,$$

and the equivalent speed is that speed₁′=E₁×speed₁.

In an embodiment, first, the heart rate-speed relationship of the user in a standard environment is obtained, then the user's first heart rate and first speed are obtained; finally, the user's equivalent speed in a standard environment is determined based on the first heart rate, the first speed, and the heart rate-speed relationship. Specifically, the user's equivalent speed in a standard environment can be determined based on the first heart rate, the first speed, and the heart rate-speed relationship; then an equivalent pace is determine based on the equivalent speed; it can be achieved by converting the equivalent speed to the corresponding equivalent pace, and the pace denotes the time taken to run per kilometer.

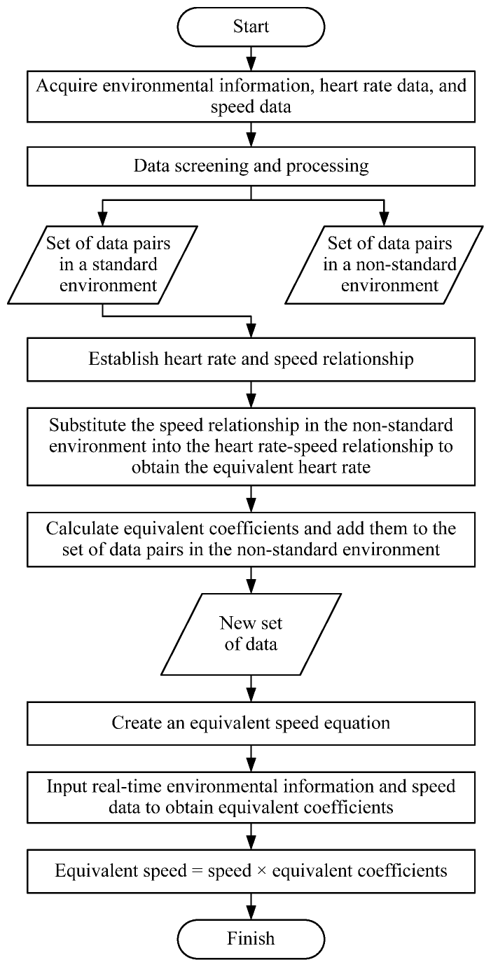

FIG. 2 is a flowchart of a method for determining an equivalent speed according to an embodiment of the present invention. The method is another possible implementation of the preceding steps. As shown in FIG. 2, the method includes the steps described below.

S210: A second heart rate and a second speed of the user are acquired in the standard environment.

The second heart rate and the second speed denote the heart rate data and speed data acquired when the user is in the standard environment.

In this embodiment, the environment in which the user is located may be considered. The heart rate data and speed data corresponding to the user in the standard environment are determined as the second heart rate and the second speed respectively when it is determined that the user is in the standard environment.

Optionally, the method also includes the steps described below. Environmental information is acquired. Whether the user is in the standard environment is determined according to the environmental information.

The second heart rate and the second speed of the user in the standard environment may be acquired by determining heart rate data and speed data corresponding to the user in the standard environment as the second heart rate and the second speed respectively when it is determined that the user is in the standard environment.

Further, the environmental information includes slope.

According to the environmental information, whether the user is in the standard environment is determined. The determination method may be determining that the user is in the standard environment when the slope is within the preset slope range.

Optionally, it may be determined according to the slope (S) whether the user is in the standard environment. The preset slope range may be −3%<S<3%. When the slope of the user's location is between −3% and 3%, it is determined that the user is in the standard environment.

Further, the environmental information includes altitude.

According to the environmental information, whether the user is in the standard environment is determined. The determination method may be determining that the user is in the standard environment when the altitude is within the preset altitude range.

Optionally, it may be determined according to the altitude (H (m)) whether the user is in the standard environment. The preset altitude range may be that H<1200 m. When the altitude of the user's location is less than 1200 m, it is determined that the user is in the standard environment.

Further, the environmental information includes temperature.

According to the environmental information, whether the user is in the standard environment is determined. The determination method may be determining that the user is in the standard environment when the temperature is within the preset temperature range.

Optionally, it may be determined according to the temperature (T (° C.)) whether the user is in the standard environment. The preset temperature range may be 10° C.≤T≤28° C. When the temperature of the user's location is between 10° C. and 28° C., it is determined that the user is in the standard environment.

Further, the temperature may denote an apparent temperature. The apparent temperature refers to the temperature equivalent converted from coldness and warmth perceived by humans, which is caused by the combined effects of air temperature, wind speed, and relative humidity. The apparent temperature is expressed in AT (° C.). Then AT=1.07T+0.2e−0.65V−2.7, and e=(RH/100)×6.105×exp(17.27T/237.7+T).

T denotes air temperature (° C.). e denotes water vapor pressure (hPa). V denotes wind speed (m/sec). RH denotes relative humidity (%).

Further, the environmental information may also be a set of two or three of slope, altitude, and temperature. For example, the environmental information may be a set of slope, altitude, and temperature of the user's location. The standard environment may be slope (S), altitude (H (m)), and temperature (T (° C.)) that satisfy the following conditions: —3%<S<3%, H<1200 m, and 10° C.≤T≤28° C. When it is determined that the environment in which the user is located meets the preceding three conditions simultaneously, the corresponding heart rate data and the speed data are determined as the second heart rate and the second speed, respectively.

S220: The heart rate-speed relationship is determined according to the second heart rate and the second speed.

In this embodiment, after the second heart rate and the second speed in the standard environment are acquired, function fitting can be performed on the second heart rate and the second speed, and then the heart rate-speed relationship can be obtained.

Optionally, the second heart rate and the second speed may be screened by using the moving window method. The time window of 1 minute is used. The data that the heart rate (HR) fluctuation does not exceed 10 bpm and the speed fluctuation does not exceed 0.5 m/s in the second heart rate and the second speed in a minute are acquired. After screening, a data pair set N consisting of the heart rate data and speed data in the standard environment is obtained. Each row in the set N is a data pair. The heart rate data and speed data are acquired at the same time.

The set N may be represented as follows:

$$N = [ \begin{matrix}{HR}_{1} & {speed}_{1} \\ … & … \\ {HR}_{n} & {speed}_{n}\end{matrix} ]$$

n is the number of data pairs.

Function fitting is performed on the data pairs in the set N to obtain the slope k and the intercept b, then the heart rate-speed relationship can be expressed as the following: HR=k×speed+b.

S230: A first heart rate and a first speed of the user are acquired. An equivalent speed of the user in the standard environment is determined according to the first heart rate, the first speed, and the heart rate-speed relationship.

In this embodiment, according to the first heart rate and the first speed acquired in real time from the user, the first speed may be substituted into the heart rate-speed relationship to obtain the equivalent heart rate in the standard environment. Then, the ratio of the first heart rate and the equivalent heart rate is used as an equivalent coefficient between the user's current environment and the standard environment. The product of the first speed and the equivalent coefficient is determined as the equivalent speed of the user in the standard environment.

Optionally, the method also includes the steps described below. Environmental information is acquired. According to the environmental information, a third heart rate and a third speed of the user in a non-standard environment are determined from the first heart rate and the first speed.

The equivalent speed of the user in the standard environment is determined according to the first heart rate, the first speed, and the heart rate-speed relationship. The determination method may be determining the equivalent speed of the user in the standard environment according to the third heart rate, the third speed, and the heart rate-speed relationship.

Specifically, the environmental information, heart rate data, and speed data may be acquired simultaneously. The third heart rate and the third speed in the non-standard environment are determined according to the environmental information. The moving window method, with 1 minute as the time window, is used to screen the non-standard environment that is stable within 1 minute (for example, the temperature fluctuation does not exceed 1° C., and the slope does not exceed 3%). On this basis, the data that the heart rate (HR) fluctuation does not exceed 10 bpm and the speed fluctuation does not exceed 0.5 m/s in the third heart rate and the third speed are screened. The data meeting the preceding requirements form a data pair set M. Each row in the set M is a data pair. The environmental information, heart rate data, and speed data are acquired simultaneously.

The set M may be represented as follows:

$$M = [ \begin{matrix}{HR}_{1} & {speed}_{1} & {T}_{1} & {S}_{1} & {H}_{1} \\ … & … & … & … & … \\ {HR}_{m} & {speed}_{m} & {T}_{m} & {S}_{m} & {H}_{m}\end{matrix} ]$$

m is the number of data pairs. T, S, and H represent temperature, slope, and altitude, respectively.

The second column in the set M, that is, the speed, is substituted into the heart rate-speed relationship, namely, HR=k×speed+b, and a set of column vectors of HR′ is obtained. The first column in the set M, that is, the heart rate (HR), is divided by the corresponding HR′, and then the heart rate equivalent coefficient E is obtained at the same speed under the temperature, slope, and altitude. E is placed in the set M′ to replace HR, and a new set is obtained.

The set M′ may be represented as follows:

$${M}^{′} = [ \begin{matrix}{E}_{1} = \frac{{HR}_{1}}{{HR}_{1}^{′}} & {speed}_{l} & {T}_{1} & {S}_{1} & {H}_{1} \\ … & … & … & … & … \\ {E}_{m} = \frac{{HR}_{m}}{{HR}_{m}} & {speed}_{m} & {T}_{m} & {S}_{m} & {H}_{m}\end{matrix} ]$$

Function fitting is performed on the data pairs in the set M′ to obtain the equivalent speed equation:

E=d(a×S²+b×S+1)^{c×speed}×T×H

a, b, c, and d are equation coefficients.

Further, after the equivalent speed equation is obtained, real-time speed, slope, temperature, and altitude can be inputted. The current equivalent coefficient E is obtained through the equivalent speed equation. The equivalent speed can be obtained by multiplying E by the current speed in the current external environment, which is equivalent to the speed in the standard environment.

Optionally, according to the environmental information, determining the third heart rate and the third speed of the user in the non-standard environment from the first heart rate and the first speed includes the steps described below. Whether the user is in a non-standard environment is determined according to the environmental information. The first heart rate and the first speed of the user in the non-standard environment are determined as the third heart rate and the third speed, respectively.

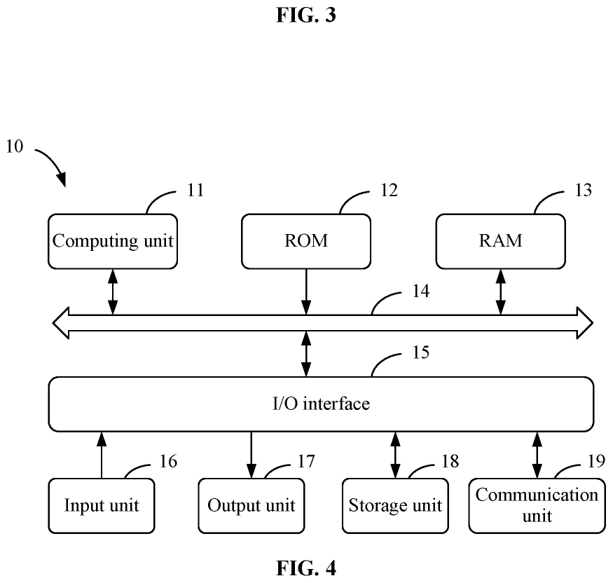

FIG. 3 is a diagram illustrating the process of a method for determining an equivalent speed according to an embodiment of the present invention. As shown in FIG. 3, environmental information, heart rate data, and speed data are acquired simultaneously, and then the data are screened and processed to obtain a data pair set N in the standard environment and a data pair set M in the non-standard environment. Function fitting is performed on the data pair set N, and the heart rate-speed relationship in the standard environment is obtained. The speed data in the data pair set M is substituted into the heart rate-speed relationship, and an equivalent heart rate HR′ is obtained. Then, an equivalent coefficient is calculated and added to the data pair set M to obtain a new set M′. Function fitting is performed on the data pairs in the set M′, and an equivalent speed equation is obtained. Real-time environmental information and speed data are inputted, and real-time equivalent coefficients are obtained. Then equivalent speed is the product of the real-time speed and the corresponding equivalent coefficients.

Optionally, the method also includes the steps described below. Environmental information is acquired. According to the environmental information, a fourth speed of the user in the standard environment is determined from the first speed. The fourth speed is determined as the equivalent speed.

Specifically, the first speed is the speed data acquired in real time for the current user. If the environment in which the user is currently located can be determined to be a standard environment according to the acquired environmental information, it is not necessary to perform the equivalent speed conversion by using the heart rate-speed relationship. The fourth speed in the standard environment can be directly determined as the equivalent speed.

Optionally, the method also includes the steps described below. Environmental information is acquired. According to the environmental information, a fourth heart rate and a fourth speed of the user in a standard environment are determined from the first heart rate and the first speed. The heart rate-speed relationship is corrected according to the fourth heart rate and the fourth speed.

Specifically, the heart rate-speed relationship of the same user may change in the standard environment. For example, the user may improve physical quality through training such as running. Then as the user's physical fitness improves, the heart rate corresponding to the same speed may decrease, so the heart rate-speed relationship in the standard environment changes. To obtain an accurate equivalent speed, the fourth heart rate and the fourth speed in the standard environment can be extracted from the first heart rate and the first speed to correct the heart rate-speed relationship. In this manner, the heart rate-speed relationship is more in line with the physical condition of the user.

Optionally, according to the environmental information, determining the fourth heart rate and the fourth speed of the user in a standard environment from the first heart rate and the first speed includes the steps described below. Whether the user is in the standard environment is determined according to the environmental information. The first heart rate and the first speed of the user in the standard environment are determined as the fourth heart rate and the fourth speed, respectively.

The method for determining an equivalent speed provided by the embodiments of the present invention converts the current speed of a user into an equivalent speed in a standard environment by using the heart rate-speed relationship in the standard environment. In this manner, the speed data conforms to the physiological intensity of the user in the current environment, the delay of changes in the human heart rate is avoided, and the user is assisted more effectively to control the intensity of exercise or change the strategy of a competition in a complex external environment.

Embodiments of the present invention also provide an apparatus for determining an equivalent speed. The apparatus includes at least one processor configured to perform the following steps:

A heart rate-speed relationship of a user in a standard environment is acquired.

A first heart rate and a first speed of the user are acquired.

An equivalent speed of the user in the standard environment is determined according to the first heart rate, the first speed, and the heart rate-speed relationship.

Optionally, the heart rate-speed relationship is preset by a system.

Optionally, when performing the step of acquiring the heart rate-speed relationship of the user in the standard environment, the at least one processor is also configured to perform the following steps:

A second heart rate and a second speed of the user in the standard environment are acquired.

The heart rate-speed relationship is determined according to the second heart rate and the second speed.

Optionally, the at least one processor is also configured to perform the following steps:

Environmental information is acquired.

According to the environmental information, whether the user is in the standard environment is determined.

When performing the step of acquiring the second heart rate and the second speed of the user in the standard environment, the at least one processor is also configured to perform the following step:

Heart rate data and speed data corresponding to the user in the standard environment are determined as the second heart rate and the second speed respectively when it is determined that the user is in the standard environment.

Optionally, the environmental information includes slope.

When performing the step of determining whether the user is in the standard environment according to the environmental information, the at least one processor is also configured to perform the following step:

It is determined that the user is in the standard environment when the slope is within the preset slope range.

Optionally, the environmental information includes altitude.

When performing the step of determining whether the user is in the standard environment according to the environmental information, the at least one processor is also configured to perform the following step:

It is determined that the user is in the standard environment when the altitude is within the preset altitude range.

Optionally, the environmental information includes temperature.

When performing the step of determining whether the user is in the standard environment according to the environmental information, the at least one processor is also configured to perform the following step:

It is determined that the user is in the standard environment when the temperature is within the preset temperature range.

Optionally, the at least one processor is also configured to perform the following steps:

Environmental information is acquired.

According to the environmental information, a third heart rate and a third speed of the user are determined in a non-standard environment from the first heart rate and the first speed.

When performing the step of determining an equivalent speed of the user in the standard environment according to the first heart rate, the first speed, and the heart rate-speed relationship, the at least one processor is also configured to perform the following step:

An equivalent speed of the user in the standard environment is determined according to the third heart rate, the third speed, and the heart rate-speed relationship.

Optionally, the at least one processor is also configured to perform the following steps:

Environmental information is acquired.

According to the environmental information, a fourth speed of the user in the standard environment is determined from the first speed.

The fourth speed is determined as the equivalent speed.

Optionally, the at least one processor is also configured to perform the following steps:

Environmental information is acquired.

According to the environmental information, a fourth heart rate and a fourth speed of the user in a standard environment are determined from the first heart rate and the first speed.

The heart rate-speed relationship is corrected according to the fourth heart rate and the fourth speed.

The apparatus for determining an equivalent speed provided by embodiments of the present invention can execute the method for determining an equivalent speed according to embodiments of the present invention and has functional modules and beneficial effects corresponding to the execution methods.

FIG. 4 is a diagram illustrating the structure of an electronic device 10 that can implement embodiments of the present invention. The electronic device is intended to represent various forms of digital computers, for example, a laptop computer, a desktop computer, a worktable, a personal digital assistant, a server, a blade server, a mainframe computer, or other applicable computers. The electronic device may also represent various forms of mobile apparatuses such as a personal digital processing apparatus, a cellular phone, a smart phone, a wearable device (for example, a helmet, glasses, a watch), and other similar computing apparatuses. The components shown herein, their connections and relationships, and their functions, are by way of examples only and are not intended to limit implementations of the inventions described and/or claimed herein.

As shown in FIG. 4, the electronic device 10 includes at least one processor 11 and a memory in a communication connection with the at least one processor 11 such as a read-only memory (ROM) 12 and a random access memory (RAM) 13. The memory stores a computer program executable by the at least one processor. The processor 11 can perform various appropriate actions and processes according to computer programs stored in a read-only memory (ROM) 12 or loaded from a storage unit 18 into a random access memory (RAM) 13. The RAM 13 can also store various programs and data required for the operation of the electronic device 10. The processor 11, the ROM 12, and the RAM 13 are connected to each other through a bus 14. An input/output (I/O) interface 15 is also connected to the bus 14.

Multiple components in the electronic device 10 are connected to the I/O interface 15, including an input unit 16, such as a keyboard or a mouse; an output unit 17, such as various types of displays or speakers; a storage unit 18, such as a magnetic disk or an optical disk; and a communication unit 19, such as a network card, a modem, or a wireless communication transceiver. The communication unit 19 allows the electronic device 10 to exchange information/data with other devices through a computer network such as the Internet and/or various telecommunication networks.

The processor 11 may be various general-purpose and/or special-purpose processing components having processing and computing capabilities. Some examples of the processor 11 include, but are not limited to, a central processing unit (CPU), a graphics processing unit (GPU), various dedicated artificial intelligence (AI) computing chips, various processors running machine learning model algorithms, a digital signal processor (DSP), and any suitable processors, controllers, and microcontrollers. The processor 11 performs various methods and processes described above, such as the method for determining an equivalent speed.

In some embodiments, the method for determining an equivalent speed may be implemented as a computer program tangibly embodied in a computer-readable storage medium such as the storage unit 18. In some embodiments, part or all of the computer program may be loaded and/or installed on the electronic device 10 via the ROM 12 and/or the communication unit 19. When the computer program is loaded into the RAM 13 and executed by the processor 11, one or more steps of the equivalent speed determination described above may be performed. Alternatively, in other embodiments, the processor 11 may be configured to perform the method for determining an equivalent speed by any other suitable means (for example, by means of firmware).

Various implementations of the systems and techniques described above herein may be implemented in digital electronic circuitry, integrated circuitry, a field programmable gate array (FPGA), an application specific integrated circuit (ASIC), an application specific standard product (ASSP), a system on chip (SOC), a complex programmable logic device (CPLD), a computer hardware, a firmware, a software, and/or combinations thereof. The various implementations may include an implementation in one or more computer programs that may be executable and/or interpretable on a programmable system including at least one programmable processor. The programmable processor may be special-purpose or general-purpose for receiving data and instructions from a memory system, at least one input apparatus, and at least one output apparatus and transmitting the data and instructions to the memory system, the at least one input apparatus, and the at least one output apparatus.

The computer program for implementing the method of the present invention may be written in any combination of one or more programming languages. These computer programs may be provided to a processor of a general-purpose computer, special-purpose computer, or other programmable data processing apparatus such that the computer programs, when executed by the processor, cause the functions/operations specified in flowcharts and/or block diagrams to be implemented. The computer program may be executed entirely or partly on the machine, as a stand-alone software package partly on the machine and partly on a remote machine, or entirely on the remote machine or server.

In the context of the present invention, a computer-readable storage medium may be a tangible medium that may contain or store a computer program for use by or in conjunction with an instruction execution system, apparatus, or device. The computer-readable storage medium may include, but is not limited to, electronic, magnetic, optical, electromagnetic, infrared, or semiconductor systems, apparatuses, devices, or any suitable combination thereof. Alternatively, the computer-readable storage medium may be a machine-readable signal medium. Concrete examples of the machine-readable storage medium may include an electrical connection based on one or more wires, a portable computer disk, a hard disk, a random access memory (RAM), a read-only memory (ROM), an erasable programmable read-only memory (EPROM or flash memory), an optical fiber, a portable compact disc read-only memory (CD-ROM), an optical storage device, a magnetic storage device, or any appropriate combination thereof.

To provide interaction with a user, the systems and techniques described herein may be implemented on an electronic device. The electronic device has a display device (for example, CRT (cathode ray tube) or LCD (liquid crystal display) monitor) for displaying information to the user; and a keyboard and pointing apparatus (for example, a mouse or a trackball) through which a user can provide input to the electronic device. Other types of apparatuses may also be used for providing interaction with a user. For example, feedback provided for the user may be sensory feedback in any form (for example, visual feedback, auditory feedback, or haptic feedback). Moreover, input from the user may be received in any form (including acoustic input, voice input, or haptic input).

In an embodiment, the processor 11 is configured to determine the equivalent pace and control the display device to show the equivalent pace.

The systems and techniques described herein may be implemented in a computing system including a back-end component (for example, a data server), a computing system including a middleware component (for example, an application server), a computing system including a front-end component (for example, a client computer having a graphical user interface or a web browser through which a user can interact with implementations of the systems and techniques described herein), or a computing system including any combination of such back-end, middleware, or front-end components. Components of a system may be interconnected by any form or medium of digital data communication (for example, a communication network). Examples of the communication network include a local area network (LAN), a wide area network (WAN), a blockchain network, and the Internet.

The computing system may include a client and a server. A client and a server are generally remote from each other and typically interact through a communication network. The relationship between the client and the server arises by virtue of computer programs running on respective computers and having a client-server relationship to each other. The server, which may be a cloud server and is also referred to as a cloud computing server or a cloud host, is a host product in a cloud computing service system. The server solves the problems of difficult management and weak service scalability in the service of a related physical host and a related VPS.

It is to be understood that various forms of processes shown above may be adopted with steps reordered, added, or deleted. For example, the steps described in the present invention may be performed in parallel, sequentially, or in different orders, as long as the desired results of the technical solutions of the present invention can be achieved, and no limitation is imposed herein.

The preceding embodiments do not limit the scope of the present invention. It is to be understood by those skilled in the art that various modifications, combinations, sub-combinations, and substitutions may be performed according to design requirements and other factors. Any modifications, equivalent substitutions, improvements, and the like made within the spirit and principle of the present invention are within the scope of the present invention.

What is claimed is:

1. A method for determining an equivalent speed, comprising:
acquiring a heart rate-speed relationship of a user in a standard environment;
acquiring a first heart rate and a first speed of the user; and
determining an equivalent speed of the user in the standard environment according to the first heart rate, the first speed, and the heart rate-speed relationship.

2. The method of claim 1, wherein the heart rate-speed relationship is preset by a system.

3. The method of claim 1, wherein acquiring the heart rate-speed relationship of the user in the standard environment, comprises:
acquiring a second heart rate and a second speed of the user in the standard environment; and
determining the heart rate-speed relationship according to the second heart rate and the second speed.

4. The method of claim 3, further comprising:
acquiring environmental information; and
determining, according to the environmental information, whether the user is in the standard environment;
wherein acquiring the second heart rate and the second speed of the user in the standard environment comprises:
determining heart rate data and speed data corresponding to the user in the standard environment as the second heart rate and the second speed respectively when it is determined that the user is in the standard environment.

5. The method of claim 4, wherein the environmental information comprises slope;
wherein determining, according to the environmental information, whether the user is in the standard environment comprises:
determining that the user is in the standard environment when the slope is within a preset slope range.

6. The method of claim 4, wherein the environmental information comprises altitude;
wherein determining, according to the environmental information, whether the user is in the standard environment comprises:
determining that the user is in the standard environment when the altitude is within a preset altitude range.

7. The method of claim 4, wherein the environmental information comprises temperature;
wherein determining, according to the environmental information, whether the user is in the standard environment comprises:
determining that the user is in the standard environment when the temperature is within a preset temperature range.

8. The method of claim 1, further comprising:
acquiring environmental information; and
determining, according to the environmental information, a third heart rate and a third speed of the user in a non-standard environment from the first heart rate and the first speed;
wherein determining the equivalent speed of the user in the standard environment according to the first heart rate, the first speed, and the heart rate-speed relationship comprises:
determining the equivalent speed of the user in the standard environment according to the third heart rate, the third speed, and the heart rate-speed relationship.

9. The method of claim 1, further comprising:
acquiring environmental information;
determining, according to the environmental information, a fourth speed of the user in the standard environment from the first speed; and
determining the fourth speed as the equivalent speed.

10. The method of claim 1, further comprising:
acquiring environmental information;
determining, according to the environmental information, a fourth heart rate and a fourth speed of the user in a standard environment from the first heart rate and the first speed; and
correcting the heart rate-speed relationship according to the fourth heart rate and the fourth speed.

11. An electronic device, comprising:
at least one processor; and
a memory in a communication connection with the at least one processor;
wherein the memory stores a computer program executable by the at least one processor, wherein the computer program is configured to, when executed by the at least one processor, cause the at least one processor to execute:
acquiring a heart rate-speed relationship of a user in a standard environment;
acquiring a first heart rate and a first speed of the user; and
determining an equivalent speed of the user in the standard environment according to the first heart rate, the first speed, and the heart rate-speed relationship.

12. A non-transitory computer-readable storage medium, which is configured to store computer instructions for implementing the method for determining an equivalent speed of claim 1 when the computer instructions are executed by a processor.
